(19) United States
(12) Patent Application Publication
(10) Pub. No.: US 2004/0077028 A1
(43) Pub. Date: Apr. 22, 2004

(54) Assay for thrombocytopenia associated with administration of platelet GPIIb-IIIa receptor antagonists

(76) Inventors: Lori A. Krueger, Carlsbad, CA (US); Alan David Michelson, Sudbury, MA (US); Andrew L. Frelinger III, North Reading, MA (US); Mark Ira Furman, Sudbury, MA (US); Marc Robert Barnard, Douglas, MA (US)

Correspondence Address: FISH & RICHARDSON PC, 225 FRANKLIN ST, BOSTON, MA, 02110, US

(21) Appl. No.: 10/644,434

(22) Filed: Aug. 20, 2003

Related U.S. Application Data
(60) Provisional application No. 60/404,811, filed on Aug. 20, 2002.

(51) Int. Cl. G01N 33/567

(52) U.S. Cl. 435/7.21

(57) ABSTRACT

Some individuals undergoing GPIIb-IIIa receptor antagonist therapy have an increased incidence of thrombocytopenia. The invention relates to an assay useful for the prediction and diagnosis of GPIIb-IIIa receptor antagonist-induced therapy.

### CROSS-REFERENCE TO RELATED APPLICATION

This application claims priority from U.S. provisional patent application serial No. 60/404,811, filed on Aug. 20, 2002, which is incorporated herein by reference in its entirety.

### TECHNICAL FIELD

This invention relates to GPIIb-IIIa receptor antagonist drug-induced thrombocytopenia or thrombotic complications.

### BACKGROUND

Platelets play an essential role in maintaining healthy vascular hemostasis. When vascular injury occurs, circulating peripheral blood platelets become activated upon exposure to sub-endothelial matrix. A hemostatic platelet plug is formed when activated platelets adhere to the damaged vessel, aggregate, and recruit additional peripheral blood platelets to the injured site. The hemostatic platelet plug is an early necessary step in progression of the coagulation cascade resulting in a stable thrombus. However, chronic inflammation of endothelium leading to persistent platelet activation, adhesion, and aggregation is a common feature of coronary artery disease. In addition, platelet aggregation may become life threatening when complete vessel blockage occurs (i.e., thrombosis) resulting in acute myocardial infarction or stroke.

Fibrinogen is the main adhesive protein responsible for mediating platelet aggregation. Platelet glycoprotein IIb-IIIa (GPIIb-IIIa, integrin α_{IIb}β₃) is the major platelet surface glycoprotein involved in ligation of the dimeric coagulation factor fibrinogen. A class of drugs known as GPIIb-IIIa receptor antagonists (e.g., abciximab, eptifibatide, and tirofiban), that block fibrinogen binding to platelet GPIIb-IIIa, reduces the incidence of life-threatening thrombosis in patients with coronary artery disease following percutaneous coronary interventions (PCI), in patients with acute myocardial infarction, and in patients in other clinical settings. However, a small but significant proportion (1-2%) of patients receiving GPIIb-IIIa receptor antagonists become thrombocytopenic.

Thrombocytopenia is a condition in which there is a decrease in the normal number of platelets in the peripheral circulation. By definition, thrombocytopenia is characterized by platelet counts of less than 100,000/μl (normal range is 150,000-400,000/μl). The condition results in the potential for increased bleeding and the decreased ability to form clots. Some individuals receiving GPIIb-IIIa receptor antagonists become profoundly thrombocytopenic (i.e., have platelet counts of less than 20,000/μl). Drug-induced thrombocytopenia can become life threatening due to the increased risk of severe bleeding and intra-cranial hemorrhage. Drug-induced thrombocytopenia also increases medical costs due to additional patient treatment, e.g., platelet transfusion and/or a prolonged hospital stay.

Of the three FDA approved GPIIb-IIIa receptor antagonists commercially available, only abciximab is a chimeric human-mouse monoclonal Fab fragment. About 6% to 7% of patients develop a human anti-chimeric antibody (HACA) after first exposure to abciximab (data on file, Centocor, Inc., Malvern, Pa. as reported by Tcheng et al., 1999, Am. Heart J. 138(1 Pt 2):S33-38). Within the patient population that develops an HACA response, the incidence of thrombocytopenia (e.g., count of <100×10⁹/L) is 3.5% and profound thrombocytopenia (e.g., a platelet count of <20×10⁹/L) occurred in less than 1% of patients (Berkowitz et al., 1998, J. Am. Coll. Cardiol. 32:311-319). Moreover, the incidence of thrombocytopenia (e.g., platelet count of <100×10⁹/L) in patients receiving abciximab for a second time (re-administration) was 3.6%, similar to that found for patients first exposed to abciximab. Thus, Berkowitz et al. report “the presence (or absence) of HACA was not predictive of the presence (or absence) of clinical efficacy, adverse events, or immune-mediated phenomena” (Tcheng et al., supra).

Current treatment for patients who become thrombocytopenic while receiving GPIIb-IIIa antagonists includes termination of GPIIb-IIIa antagonist therapy. Since patients receiving GPIIb-IIIa antagonists are also exposed to other drugs that can cause thrombocytopenia (e.g., heparin) they may be deprived of the benefits of GPIIb-IIIa antagonists without cause. Thus, there exists a need for correctly identifying the underlying cause of thrombocytopenia so that appropriate treatment may be pursued.

The incidence of thrombocytopenia and the increased associated health risks in patients receiving GPIIb-IIIa receptor antagonist therapy indicates a need for a diagnostic assay to identify individuals who have developed thrombocytopenia or thrombotic complications in response to the treatment and a predictive assay to identify individuals likely to develop thrombocytopenia or thrombotic complications when receiving such treatment.

### SUMMARY

The invention is based, in part, on the discovery that some murine monoclonal antibodies specific for epitopes on platelet surface GPIIb-IIIa can augment platelet activation in the presence of low dose (sub-maximal) platelet agonist and therapeutic levels of GPIIb-IIIa receptor antagonists. This activation appears to be platelet FcγRII (CD32) receptor mediated, as evidenced by the ability of FcγRII receptor blocking antibodies to inhibit the enhanced platelet response under these conditions. These features are used in assays for predicting thrombocytopenia, diagnosing thrombocytopenia, or increased risk of thrombotic complications associated with GPIIb-IIIa receptor antagonist therapy.

Accordingly, the invention includes a method of determining whether an individual (e.g., a human) has thrombocytopenia or is at risk for developing thrombocytopenia as a result of GPIIb-IIIa receptor antagonist treatment. The method includes obtaining a sample that contains at least serum or plasma from the individual and platelets, adding a GPIIb-IIIa receptor antagonist (e.g., abciximab, eptifibatide, or tirofiban) to the sample to form an antagonist mixture; adding a submaximal concentration of a platelet agonist (e.g., adenosine diphosphate (ADP), thrombin receptor activating peptide (TRAP), iso-TRAP, or collagen) to the antagonist mixture to form an assay solution; and assaying platelet activation in the assay solution; wherein an increase in the amount of platelet activation in the assay solution compared to a reference indicates that the individual has thrombocytopenia or is at risk for developing thrombocytopenia as a result of GPIIb-IIIa receptor antagonist treatment. The platelets can be from the individual (e.g., from a whole blood sample or a plasma sample) or from a donor. In general, if the platelets are from a donor, the donor is an ABO-compatible donor. In some embodiments, a CD32 blocking antibody is added to the sample prior to adding GPIIb-IIIa receptor antagonist, and an increase in platelet activation in the sample is blocked by adding the CD32 blocking antibody, thus indicating the presence of pathologic anti-platelet antibodies.

In some embodiments of the invention, the method of detecting platelet activation includes detecting platelet surface P-selectin, phosphatidylserine, or leukocyte-platelet aggregates. Flow cytometry can be used to detect platelet activation.

In other embodiments of the invention, the reagent used to detect platelet activation is added before the GPIIb-IIIa antagonist. Alternatively, the reagent used to detect platelet activation is added after the GPIIb-IIIa antagonist and before the platelet agonist, is added with the GPIIb-IIIa antagonist, or is added with the platelet agonist.

The method can include determining the FcγRII genotype of platelets used in the assay.

A “reference” is used for comparing the results of an assay and provides a standard value. The reference can be a predetermined number, condition, or numerical range. In some embodiments of the invention, the reference is a control. In a control, a subject's sample is assayed both in the presence and absence of a component of the assay method. For example, a subject's sample can be assayed in the presence and absence of a GPIIb-IIIa receptor antagonist. In some embodiments, a control is provided by assaying a sample from an individual who does not have thrombocytopenia or thrombotic complications, or any apparent disorder related to platelets. In general, the control sample has the same FcγRII receptor genotype as the subject's sample (test sample).

Without committing to any particular theory, it may be that the augmentation of platelet activation exhibited by murine monoclonal antibodies that specifically bind to epitopes on platelet surface GPIIb-IIIa mimic the action of pre-existing autoantibodies that are present in some patients who develop thrombocytopenia or thrombotic complications after receiving GPIIb-IIIa receptor antagonist therapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF DRAWINGS

FIG. 1 is a bar graph depicting the results of experiments in which neutrophil-platelet aggregates were measured in the presence or absence of GPIIb-IIIa receptor antagonists.

FIG. 2 is a bar graph depicting the results of experiments in which platelet surface P-selectin expression was assayed in the presence of ADP and in the presence or absence of the mouse monoclonal antibody 5B12 (CD41), and in the presence or absence of abciximab.

FIG. 3 is a bar graph depicting the results of experiments in which platelet surface P-selectin expression was assayed in the presence of ADP; in the presence or absence of mouse monoclonal antibody Y2/51; in the presence or absence of abciximab, tirofiban, or eptifibatide; and in the presence or absence of an FcγRII (CD32) receptor-blocking antibody (Mab IV.3).

### DETAILED DESCRIPTION

The present invention relates to methods for predicting the risk of or diagnosing existing thrombocytopenia or thrombotic complications associated with therapeutic administration of platelet GPIIb-IIIa receptor antagonist drugs. The invention includes assays that, by testing a sample from an individual (using serum, plasma, or other suitable preparation), are useful for identifying those individuals, prior to treatment with GPIIb-IIIa receptor antagonists, who are at risk for becoming thrombocytopenic or who are at risk for developing thrombotic complications as a result of platelet GPIIb-IIIa receptor antagonist therapy. The invention also encompasses assays for diagnosing individuals (subjects) who are thrombocytopenic or have developed thrombotic complications as a result of platelet GPIIb-IIIa receptor antagonist therapy. Such individuals include those who are currently receiving or have recently received platelet GPIIb-IIIa receptor antagonist therapy. In these individuals, the assay is useful for determining whether their thrombocytopenia or thrombotic condition is related to the GPIIb-IIIa receptor antagonist therapy or some other condition or therapy. Thus, the assay is also useful for determining the course of therapy to be provided to individuals for conditions in which GPIIb-IIIa receptor antagonist therapy is an option, e.g., coronary artery disease and related disorders such as stroke and acute myocardial infarction.

In general, the methods of the invention include 1) obtaining from a subject a blood sample containing all plasma and cellular components, obtaining from a subject platelet rich plasma, or obtaining from a subject platelet poor plasma or serum and mixing the platelet poor plasma or serum with ABO-compatible donor platelets to form a mixture; 2) adding a GPIIb-IIIa receptor antagonist to the sample or mixture to form an antagonist mixture; 3) adding a sub-maximal concentration of platelet agonist to the antagonist mixture to form an assay solution; and 4) assaying platelet activation in the assay solution. Platelet activation can be assessed, e.g., using fluorescently conjugated reagents such as monoclonal antibody reagents or ligands and flow cytometry to detect an indicator of platelet activation such as platelet surface P-selectin, platelet surface phosphatidylserine expression, or leukocyte-platelet aggregates. In the presence of a GPIIb-IIIa receptor antagonist and a sub-maximal concentration of a platelet agonist, an increase in platelet activation (i.e., an increase in platelet activation markers) compared to platelet activation in the absence of the GPIIb-IIIa receptor antagonist indicates that the subject has, or has an increased risk of developing, thrombocytopenia or is experiencing thrombotic episodes as a result of GPIIb-IIIa receptor antagonist therapy.

Fcγ-receptors (FcγR) recognize the Fc portion of IgG. The only FcγR on the platelet surface is FcγRIIa (CD32). Monoclonal antibodies specific for CD32 recognize and block the function of the FcγRIIa receptor. In some embodiments of the invention, an FcγRIIa blocking step is included in the assay (Technique 4). In this case, the assay is performed in the presence (test sample) and absence (control sample) of a blocking concentration of, e.g., an FcγRIIa receptor antibody (a blocking antibody), for example, antibody Mab IV.3 (Medarex, Princeton, N.J.), which is directed against the FcγRIIa receptor on the platelet surface (Tomiyama et al., 1992, Blood, 80:2261-2268). The results of such an assay are shown in Example 2. Performing the assay in the presence and absence of a blocking antibody allows one to distinguish between Fc-mediated and other mechanisms of platelet destruction such as complement-mediated destruction. The blocking antibody is typically added to the sample before the GPIIb-IIIa receptor antagonist. The absence of platelet activation in the presence of an agent that blocks the FcγRII receptor indicates that the presence of, or the predisposition to, thrombocytopenia or thrombotic complications is immune (e.g., autoimmune) mediated.

The degree of enhanced platelet activation also corresponds to the AA131 FcγRII genotype of the platelet donor. Different genotypes of the receptor are associated with different levels of enhancement. The FcγRII receptor can have an amino acid substitution (arginine or histidine) at position AA131 resulting in two alleles and three genotypes; R/R131, R/H131, or H/H131. The greatest enhancement of platelet activation is observed with platelets from R/R131 FcγRII subjects followed by platelets from R/H131 FcγRII subjects. Samples from H/H131 subjects have the lowest levels of enhanced platelet activation in the presence of therapeutic concentrations of GPIIb-IIIa receptor antagonists and sub-maximal concentrations of platelet agonists. The number of platelet surface FcγRIIa receptors influences the extent of enhanced platelet activation (Tomiyama, 1992, Blood 80:2261-2268). This effect is most pronounced in subjects with R/R131 and R/H131 genotypes. It has been shown that the FcγRII genotype may influence the potential risk of developing pathological conditions such as autoimmune disorders (van der Pol, 1998, Immunogenetics 48:222-232). Therefore, information regarding a subject's FcγRIIa receptor AA131 genotype and platelet surface receptor density provides additional information, e.g., for predicting the clinical outcome of providing that subject with GPIIb-IIIa receptor agonist therapy. Accordingly, the new methods can include the step of determining the subject's FcγRII receptor genotype, FcγRII phenotype, or quantitating platelet surface FcγRIIa receptor density. In general, if platelets from a heterologous donor are used in an assay, the platelets are from a donor of the same genotype as the subject being tested. Also, if platelets used in an assay as a reference or control are from an individual (or is pooled from more than one individual) other than the subject, the control or reference platelets are generally matched in genotype to the subject being tested.

Methods of Assaying Platelet Activation

The new methods measure platelet activation with a subject's blood sample in the presence of a GPIIb-IIIa receptor antagonist. Platelet activation can be measured using methods known in the art. For example, flow cytometry can be used to assay for platelet-derived microparticle formation (Lee et al., 1996, Br. J. Haematol. 95:724-731).

Platelet activation can also be assayed using flow cytometry to detect P-selectin (CD62P; e.g., Technique 1 (infra), McEver, 2001, Platelets, ed. Alan D. Michelson, Academic Press, New York), phosphatidylserine (e.g., by annexin V binding; Technique 2 (infra), Furman, 2000, Thromb. Haemost., 84:492-498), or leukocyte-platelet aggregates (Technique 3 (infra), Michelson, 2001, Circulation 104:1533-1537)

The methods can also be used to test the likelihood that any GPIIb-IIIa receptor antagonist will cause undesirable amounts of platelet activation in a subject. This can be useful, e.g., for testing new GPIIb-IIIa receptor antagonists prior to including them in clinical trials.

Determining a Sub-Maximal Concentration of Platelet Agonist

The new methods include a step of adding a sub-maximal concentration of a platelet agonist to the test and control mixtures. Platelet agonists include ADP (adenosine diphosphate), TRAP (SFLLRN; thrombin receptor activating peptide) or iso-TRAP (iso(S)FLLRN), or collagen. A sub-maximal concentration of platelet agonist is defined as an agonist concentration resulting in platelet activation that is less than maximal platelet activation. The degree of activation is determined using a specific marker of platelet activation such as platelet surface P-selectin expression, leukocyte-platelet aggregation, or platelet surface phosphatidylserine expression. A sub-maximal dose of platelet agonist is determined by performing an agonist dose response curve. Such methods are known in the art.

The present methods are based on augmentation of platelet activation in response to a sub-maximal concentration of agonist in the presence of a GPIIb-IIIa receptor antagonist. A sub-maximal agonist dose near or slightly less than the EC50 is optimal. A dose-response curve can be generated to determine the optimal sub-maximal agonist concentration. For example, a sub-maximal dose of ADP can range from a concentration of about 0.05 μM to about 5.0 μM ADP and is typically a concentration of about 0.5 μM. For iso-TRAP, a range of 0.1 μM to 15 μM is sub-maximal. Typically a sub-maximal concentration is about 1.5-2.0 μM. For TRAP, a range of about 0.5-15 μM is sub-maximal, and a typical sub-maximal concentration is about 5 μM.

Determination of Optimal Antibody or Ligand Concentration for Use in Assays

Optimal antibody or ligand concentrations are determined using methods known in the art. In general, cells with high levels of antigen expression are prepared using the sample preparation technique described herein (i.e., for determination of platelet surface P-selectin expression, platelet surface phosphatidylserine expression, or leukocyte-platelet aggregates). In general, it is desirable to use an antibody or ligand concentration that maximizes the difference between a positively labeled sample and a negative control, such as an isotype control of similar antibody or ligand concentration, fluorochrome and F:P (fluorochrome to protein) ratio, or a sample in which the specific reagent target is not expressed or blocked. Both the negative control and positively labeled fluorescence distribution should be on the same scale (i.e., fall within the four-decade logarithmic scale of the fluorescence histogram generated by flow cytometry). Depending on the antibody or ligand reagent, receptor of interest, and assay technique being used, typical reagent concentrations vary between 0.1-20.0 μg/ml.

Whole Blood Samples

In general, peripheral blood is drawn from a subject (such as an individual who is a candidate for GPIIb-IIIa receptor antagonist therapy) into a suitable anticoagulant. Anticoagulants include 3.2% or 3.8% sodium citrate anticoagulant or PPACK (D-phenylalanyl L-prolyl-L-arginine chloromethyl ketone; a direct thrombin inhibitor; Greiner Vacuette, Monroe, N.C.). The whole blood sample is incubated (e.g., for 20 minutes) at between about 22° C. and 37° C. with a therapeutic concentration of a GPIIb-IIIa receptor antagonist, e.g., 10 μg/ml abciximab (ReoPro®, Centocor, Malvern, Pa.), 1 μg/ml eptifibatide (Integrilin®, COR Therapeutics, South San Francisco, Calif.), or 50 ng/ml tirofiban (Aggrastat®, Merck & Co, West Point, Pa.). Control samples contain buffer instead of GPIIb-IIIa receptor antagonist. Platelet-rich plasma (PRP), prepared using methods known in the art or as described herein and incubated with a GPIIb-IIIa antagonist as described herein, can be used as an alternative to whole blood in the assays described herein.

Preparation of Platelet Rich Plasma (PRP)

Platelet-rich plasma (PRP) can be prepared using methods known in the art. For example, within 30 minutes of drawing blood, the whole blood is centrifuged at 150-200×g for 10-15 minutes. The supernatant (PRP) is removed to a clean polypropylene or siliconized glass test tube without disturbing the buffy coat and red cell layers. PRP from a subject can be used, e.g., instead of whole blood in assays described herein.

Preparation of Platelet Poor Plasma (PPP)

Platelet-poor plasma (PPP) is prepared using methods known in the art. For example, within 30 minutes of drawing blood, the whole blood is centrifuged at 800×g for 10-15 minutes. The supernatant constitutes the PPP which is removed to a clean polypropylene or siliconized glass test tube, without disturbing the buffy coat and red cell layers. Platelet-poor plasma should be essentially free of platelets.

PPP prepared from a subject can be used, e.g., in assays in which allogeneic platelets are added to PPP to generated reconstituted PRP. In general, PPP from a subject is prepared if an assay is not to be performed soon after a blood sample is withdrawn from the subject (e.g., if the sample is to be frozen before use).

Assays

The platelet activation assays to be used for the prediction of subjects at risk or diagnosis of thrombocytopenic or development or thrombotic complications as a result of platelet GPIIb-IIIa receptor antagonist therapy are further described in the Techniques infra. In general, the assay methods are as follows: 1) platelet surface P-selectin expression (Technique 1, below), 2) platelet surface phosphatidylserine expression (Technique 2), or 3) leukocyte-platelet aggregates (Technique 3). Variations on these three indicators of platelet activation are: a) inclusion of FcγRIIa blocking antibody (Technique 4), and/or b) if subject's autologous platelets are not available, utilization of donor platelets (Technique 5) of a known FcγRIIa receptor genotype or phenotype or known to exhibit enhanced activation in the presence of sub-maximal platelet agonist, GPIIb-IIIa receptor antagonist and GPIIb-IIIa receptor specific monoclonal antibodies e.g., Y2/51 or 5B12. Since the FcγRIIa phenotype has known pathologic implications in immune mediated processes, genotyping subjects for FcγRIIa can be used to supplement the platelet assay. Platelet responsiveness to immunoglobulin is influenced in part by FcγRIIa phenotype. Therefore, in some embodiments of the method, particularly those in which allogeneic platelets are used with plasma (or serum) from a subject, the FcγRII genotype or phenotype of the donor platelets is identified (Technique 6).

Technique 1: Assay for Detecting Platelet Activation Using Surface P-Selectin in Whole Blood or PRP

After a subject's blood sample has been incubated with a GPIIb-IIIa antagonist as described above, it is diluted (e.g., between 1:10 and 1:15) in autologous platelet-poor plasma (PPP). Platelet activation is assayed. Briefly, an aliquot (e.g., 20 μl) of dilute whole blood (or PRP)-GPIIb-IIIa receptor antagonist mix is incubated for 15-30 minutes at an appropriate temperature (for example, between about 22° C. and 37° C.) with sub-maximal concentration of a platelet agonist (e.g., in 10 μl). Examples of sub-maximal concentrations of platelet agonist are 0.5 μM ADP, 5.0 μM thrombin receptor activating peptide (TRAP), or 1.5 μM iso-TRAP. ADP is not generally used in the assay when the subject is receiving ADP receptor antagonist therapy. To identify platelets and to detect platelet activation, detection reagents are added. These include an appropriate concentration of a fluorescently conjugated monoclonal antibody (or reagent) that specifically binds to a marker of platelet activation (e.g., binds to P-selectin, CD62P) and a platelet identifier that does not interfere with binding of the reagent used to detect platelet activation, e.g., an antibody that specifically binds to CD41, CD61, CD42a, or CD42b (e.g., BD Pharmingen, San Jose, Calif., DAKO, Carpinteria, Calif., Beckman-Coulter Immunotech, Miami, Fla.) are added to the assay mixture.

Fab fragments derived from platelet-specific antibodies can also be used. The use of Fab fragments avoids the monoclonal antibody induced FcγRIIa activation that occurs in the presence of Y2/51, low dose platelet agonist, and some GPIIb-IIIa receptor antagonists. The detection reagents can be added to the assay mixture before adding the platelet agonist, simultaneously with platelet agonist, after platelet agonist, or after fixation (see below). The fluorescent conjugates of the platelet activation and platelet identifier reagents are distinct from each other (e.g., fluorescein and phycoerythrin) so that each can be detected, e.g., using flow cytometry. Methods for determining optimal (e.g., saturating) concentrations of monoclonal antibody and reagents are known in the art and are discussed herein.

After incubation with platelet agonist and antibody, the samples are fixed, e.g., with about 10-20 times the assay volume in 1% formalin (e.g., methanol free, ultrapure, Polysciences, Warrington, Pa.) for about 15 minutes at room temperature. The samples are stored at 4° C. until analysis by flow cytometry. Other appropriate fixatives can be used.

The samples are generally analyzed using flow cytometry. Platelets are identified by light scatter characteristics and labeling of platelets using platelet-specific antibodies (e.g., antibodies that recognize CD41, CD61, CD42a, or CD42b). The binding of antibodies directed against indicators of platelet activation (e.g., anti-P-selectin, CD62P) is assayed using flow cytometry. The data are displayed in fluorescence histograms gated on platelet events. The mean fluorescence intensity and the percent positive (above background) measurements are recorded.

The relative amount of fluorescence (or percent positive above background) in the subject's sample is compared to a control or reference sample as described herein. For example, a reference can be a sample that is prepared as described in this Technique without GPIIb-IIIa antagonist. A reference can also be established in which a previously determined level of platelet activation (e.g., as measured by flow cytometry to detect an indicator of platelet activation) used for comparison to the level of platelet activation in the sample.

An increase in the amount of fluorescence (or percent positive above background) in the subject's sample compared to the reference indicates an increased amount of platelet activation in the subject's sample.

Thus, the presence of a condition such as autoantibodies in a subject's sample that can induce thrombocytopenia or thrombotic complications in the presence of a specific GPIIb-IIIa receptor antagonist is diagnosed when there is an increase in platelet activation markers (e.g., platelet surface P-selectin expression), in the presence of a specific GPIIb-IIIa receptor antagonist and a sub-maximal dose of platelet agonist compared to the amount of platelet activation observed in the absence of the specific GPIIb-IIIa receptor antagonist.

Technique 2: Assays Detecting Pro-Coagulant Platelet-Derived Microparticles

The methods include assays in which platelet activation is detected by assaying pro-coagulant platelet-derived microparticles. In such an assay, peripheral blood is drawn from a subject into a suitable anticoagulant such as 3.2% sodium citrate anticoagulant or PPACK. After the blood is drawn from a subject (e.g., within 20 minutes), the subject's whole blood or platelet-rich plasma is incubated (e.g., for about 20 minutes at about 22° C.-37° C.) with a GPIIb-IIIa receptor antagonist at a therapeutic concentration (e.g., 10 μg/ml abciximab, 1 μg/ml eptifibatide, or 50 ng/ml tirofiban). Control samples do not contain receptor antagonist. The sample is subsequently diluted between about 1:5 to 1:10 in autologous platelet-poor plasma. The diluted sample is then diluted 1:10 in buffer containing 5 mM GPRP (tetra-peptide glycine-proline-arginine-proline; U.S. Pat. No. 5,246,832). Typically, 10 μl of whole blood and 90 μl Hepes-Tyrodes buffer/GPRP are used. GPRP is used to prevent fibrin/fibrinogen polymerization that occurs in the presence of thrombin and can occur upon buffer recalcification. Recalcification occurs in the next step of the assay.

The diluted whole blood (dWB) or diluted PRP (dPRP) sample containing GPRP is immediately incubated 1:1 with an agonist (for example, 20 μM collagen prepared in buffer supplemented with 6 mM Ca²⁺ (3 mM final concentration) and additional 5 mM GPRP to a final concentration of 2.5 mM. Reference samples are incubated in buffer with agonist (e.g., buffer containing 6 mM Ca²⁺ and 5 mM GPRP). For example, the mixture (e.g., 15 μl dWB or dPRP and 15 μl agonist solution or buffer alone is incubated at about 22° C.-37° C. for about 10-60 minutes. After this incubation, the sample is labeled with monoclonal antibody-annexin V cocktail (e.g., in 10 μl). The antibody cocktail includes fluorescently conjugated annexin V reagent that specifically binds to phosphatidylserine (B.D. Pharmingen, St. Jose, Calif.) and a fluorescently conjugated platelet specific monoclonal antibody such as one that specifically binds to CD42a, CD42b, CD41, or CD61. The incubations are typically performed at room temperature for about 10-60 minutes. The fluorescent conjugates of the reagents are distinguishable from each other (e.g., fluorescein and phycoerythrin) so that both can be detected using flow cytometry. Determination of optimal concentrations of monoclonal antibody and reagents (such as Annexin V) is discussed supra.

The labeled sample is fixed in 10-20 times the total assay volume (e.g., 400-800 μl) in 1% formalin solution, e.g., at room temperature for about 15 minutes. The labeled samples are stored at 4° C. prior to analysis

The labeled samples are analyzed using fluorescence-activated flow cytometry. Platelets can be identified by characteristic light scatter alone, or by light scatter and positive labeling with a platelet-specific monoclonal antibody (e.g., on that specifically binds to CD41, CD61, CD42a or CD42b). Annexin-V labeling is displayed on fluorescence histograms gated on platelet events (Furman, 2000, Thromb. Haemost. 84:492-498). Both the mean fluorescence intensity and the percent positive (above background) measurements are recorded. The relative amount of fluorescence (or percent positive above background) in the subject's sample is compared to a reference. An increase in the amount of fluorescence (or percent positive above background) in the subject's sample compared to the reference indicates an increased amount of platelet activation in the subject's sample.

Thus, the presence of a condition such as autoantibodies in a subject's sample that can induce thrombocytopenia or thrombotic complications in the presence of a specific GPIIb-IIIa receptor antagonist is diagnosed when there is an increase in platelet activation markers (e.g., procoagulant platelet-derived microparticles), in the presence of a specific GPIIb-IIIa receptor antagonist and a sub-maximal dose of platelet agonist compared to the amount of platelet activation observed in the absence of the specific GPIIb-IIIa receptor antagonist.

Technique 3: Assays Detecting Leukocyte-Platelet Aggregates

In some embodiments, platelet activation is detected by assaying leukocyte-platelet aggregation. In such protocols, peripheral blood is drawn from a subject into a suitable anticoagulant, for example 3.2% sodium citrate anticoagulant or PPACK. After the blood is drawn (e.g., within 20 minutes) the subject's whole blood is incubated (e.g., for about 20 minutes at an appropriate temperature (e.g., about 22° C.-37° C.) with a GPIIb-IIIa receptor antagonist at a therapeutic concentration (e.g., 10 μg/ml abciximab, 1 μg/ml eptifibatide, or 50 ng/ml tirofiban). Control samples do not contain receptor antagonist.

An aliquot (e.g., 20 μl) of whole blood-GPIIb-IIIa mix is incubated for 15-30 minutes at an appropriate temperature (for example, between about 22° C. and 37° C.) with sub-maximal doses of a platelet agonist (e.g., in 10 μl), e.g., 0.5 μM ADP, 5.0 μM thrombin receptor activating peptide (TRAP), or 1.5 EM iso-TRAP. ADP is not generally used in the assay when the subject is receiving ADP receptor antagonist therapy. Concentrations of a platelet-specific fluorescently conjugated monoclonal antibody (or reagent) and a leukocyte-specific identifier (e.g., an antibody that specifically binds to CD14 (monocyte-specific), CD45 (pan-leukocyte marker), or CD64 (which is constitutively expressed on monocytes and on activated neutrophils)) sufficient to detect platelets and leukocytes in the sample are also included in the assay mix. These reagents can be added to the sample prior to platelet agonist, simultaneously with platelet agonist, after platelet agonist, or after fixation and red cell lysis (see below). The fluorescent conjugates of the reagents are labeled to be distinguishable from each other (e.g., fluorescein and phycoerythrin) so that both can be detected by flow cytometry. Determination of optimal (e.g., saturating) concentrations of monoclonal antibody and reagents is discussed supra.

The leukocytes are fixed and the erythrocytes lysed, for example, by adding a 1:1 volume of 1.5× Hank's Balanced Salt Solution (HBSS) and 1% formalin for 10 minutes at 22° C. followed by addition of a 10× volume of water (Michelson, et al., 2001 supra). Other methods of fixation and red cell lysis can be used.

Samples are generally analyzed by flow cytometry. Leukocyte-specific monoclonal antibody labeling of cells may be used in combination with light scatter to identify specific leukocyte populations (e.g., monocyte, neutrophil, and lymphocyte populations). Leukocyte-platelet aggregates are identified using fluorescence histograms displaying fluorescence (above background) of platelet specific markers (e.g., CD41, CD61, CD42a or CD42b) from the gated monocyte, neutrophil or lymphocyte populations.

Both the mean fluorescence intensity and the percent positive (above background) measurements are recorded. The relative amount of fluorescence (or percent positive above background) in the subject's sample is compared to a reference. An increase in the amount of fluorescence (or percent positive above background) in the subject's sample compared to the reference indicates an increased amount of platelet activation in the subject's sample.

The presence of a condition such as autoantibodies in a subject's sample that can induce thrombocytopenia or thrombotic complications in the presence of a specific GPIIb-IIIa receptor antagonist is diagnosed when there is an increase in platelet activation markers (e.g., leukocyte-platelet aggregates), in the presence of a specific GPIIb-IIIa receptor antagonist and a sub-maximal dose of platelet agonist compared to the amount of platelet activation observed in the absence of the specific GPIIb-IIIa receptor antagonist.

Platelet aggregation can be assayed using other methods known in the art, e.g., Tomiyama et al., 1992, supra.

These methods are further illustrated by Examples 1 and 2 (infra).

Technique 4: Assay for Individuals at Risk of Becoming Thrombocytopenic or Developing Thrombotic Complications as a Result of Platelet GPIIb-IIIa Receptor Antagonist Therapy Using an FcγRII-Specific Reagent

In some embodiments, assays can include incubating the subject's whole blood or PRP in the presence of blocking concentrations of a FcγRIIa-specific reagent (e.g., CD32-specific monoclonal antibody) prior to the addition of GPIIb-IIIa receptor antagonist. This variation of the assay (e.g., the assays described in Techniques 1-3, supra) is used to differentiate between FcγRII-mediated immune platelet activation and other mechanisms of platelet destruction (i.e., complement-mediated). In this variation of the invention, an aliquot of the subject's sample is incubated for 10-30 minutes at 22° C.-37° C. in the presence of the FcγRII-specific blocking reagent (e.g., mouse monoclonal CD32 specific antibody IV.3 at 10 μg/ml). Another aliquot is prepared that does not contain the FcγRII-specific blocking reagent.

Following incubation in the FcγRII-specific reagent, the assays are carried out as described above in Technique 1, Technique 2, or Technique 3. The method can be performed using a fresh sample from the subject that contains the subject's platelets (as described supra) or using allogeneic donor platelets (as described infra). Example 3 illustrates such an assay.

Activation in the presence of FcγRII-specific blocking reagent indicates that the observed platelet activation is not mediated by FcγRII, and is therefore not likely to be mediated by an autoantibody.

Technique 5: Assay for Individuals at Risk of Becoming Thrombocytopenic or Developing Thrombotic Complications as a Result of Platelet GPIIb-IIIa Receptor Antagonist Therapy Using Allogeneic Platelets

In general, testing for susceptibility to thrombocytopenia or thrombotic complications resulting from GPIIb-IIIa receptor antagonist therapy is performed using a subject's own freshly drawn blood. However, when fresh blood from the subject is not available, the assays (e.g., as in Technique 1, Technique 2, and Technique 4) can be performed using fresh, allogeneic ABO-compatible donor platelets. The method is similar to that described in Technique 1 and Technique 2, except that the subject's platelet poor plasma and allogeneic ABO-compatible donor platelets are mixed and used in the assay instead of subject's whole blood or PRP.

In this variation of an assay, blood is drawn from the subject into anticoagulant as described above. Platelet poor plasma (PPP) is then generated by centrifuging the anti-coagulated whole blood as described supra. The PPP is frozen immediately after centrifugation and thawed just prior to testing. If PPP from the subject is not available, serum from the subject can be used.

To assay for susceptibility to thrombocytopenia or thrombotic complications, thawed PPP is mixed with isolated allogeneic platelets from ABO-compatible donors of known FcγRII AA131 genotype. For example, a 1:10 to 1:15 mix of allogeneic platelets and donor plasma is used. The resulting sample comprising PPP from the subject and allogeneic platelets is “reconstituted PRP.” The reconstituted PRP is then tested as described in, e.g., Technique 1 or Technique 2. Briefly, reconstituted PRP is incubated (e.g., for 20 minutes) at between about 22° C. and 37° C. with a therapeutic concentration of a GPIIb-IIIa receptor antagonist, e.g., 10 μg/ml abciximab (ReoPro®, Centocor, Malvern, Pa.), 1 μg/ml eptifibatide (Integrilin, Cor Therapeutics, South San Francisco, Calif.) or 50 ng/ml tirofiban (Aggrastat®, Merck & Co, West Point, Pa.). Control samples contain buffer instead of GPIIb-IIIa receptor antagonist. The assay methods described in, e.g., Technique 1 or Example 2 can then be followed.

The initial whole blood dilution in PPP step described in Technique 1 and Technique 2 is not used in this variation of the assay because dilution is essentially performed by preparing the reconstituted PRP. The leukocyte-platelet aggregate variation of the assay (Technique 3) cannot be performed using PPP because the allogeneic platelets are separated from the other cellular components (leukocytes) during the generation of PRP.

In some cases, the FcγRIIa genotype of the allogeneic donor platelets is determined. Platelet reactivity depends, in part, on the FcγRII genotype. The ability of the receptor to induce platelet activation also depends on the number of surface FcγRIIa receptors per platelet. AA131 Arg/Arg alleles are most sensitive to activation by the most common IgG isotypes followed by AA131 Arg/His and least reactive is the AA131 His/His genotype. However, only AA131 His/His and AA131 Arg/His can recognize the Fc portion of IgG2 isotype. The isotype of the putative pathological antibody responsible for GPIIb-IIIa receptor antagonist induced thrombocytopenia (or thrombotic complications) is not known. Therefore, to maximize the possibility of detecting pathological antibodies of any IgG isotype in a subject's plasma, the AA131 genotype of donor platelets is determined. Donor platelets with both AA131 His and Arg alleles are used in this variation of the assay. Alternatively, donor platelets shown to express increased markers of platelet activation in the presence of GPIIb-IIIa receptor antagonists, sub-maximal platelet agonists and monoclonal antibodies Y2/51 or 5B12 as (e.g., as described supra) can be used.

Technique 6: Assay of FcγRII Receptor

The allelic composition of the AA131 FcγRIIa receptor on platelets affects the reactivity of the receptor immunoglobulin (Brandt et al., 1995, Thrombosis and Haemostasis 74:1564-1572). In addition, the number of platelet surface FcγRIIa receptors plays a role in platelet activity. As discussed above, platelet activation is the lowest in His/His individuals and highest in Arg/Arg individuals with heterozygotes displaying an intermediate amount of responsiveness. Fc receptor-mediated platelet activation is well described in heparin-induced thrombocytopenia. Knowledge of a subject's FcγRII receptor genotype provides information useful for predicting whether a subject is at risk for developing thrombocytopenia or thrombotic complications.

To assay FcγRIIa receptor genotype, buffy coats prepared from the subject's peripheral blood sample is preserved (e.g., by freezing) for FcγRII receptor genotyping. The allelic composition of the sample is determined using methods known in the art, e.g., using SNP-PCR or phenotyping (see Bachelot et al., 1995, Thrombosis and Haemostasis 74:1557-1563; Brandt et al., 1995, supra). In some cases, the total number of FcγRII sites on the platelet surface is quantitated.

This invention is further illustrated by the following examples which should not be construed as limiting.

### EXAMPLES

### Example 1

### Assay Detecting Leukocyte-Platelet Aggregates

Experiments were performed to demonstrate some of the assay methods described herein and to investigate a possible mechanism by which serum augments platelet activation in the presence of a GPIIb-IIIa receptor antagonist.

In these experiments, CD61 (GPIIIa)-specific monoclonal antibody Y2/51 (DAKO) was tested for its ability to augment platelet activation using an assay of leukocyte-platelet aggregates in the presence of GPIIb-IIIa antagonists. Three antagonists were tested; abciximab (6.5 μg/ml), tirofiban (50 ng/ml), and eptifibatide (1 mg/ml). The assays were performed as described herein for detection of neutrophil-platelet aggregates (Technique 3, supra). Briefly, citrate anticoagulated whole blood was incubated with and without each of the GPIIb-IIIa receptor antagonists, activated with a sub-maximal (low) dose of ATP (0.5 μM), and labeled with FITC conjugated Y2/51 (an anti-CD61 antibody; DAKO) antibodies. The samples were then fixed with 1% formalin. Neutrophil-platelet aggregates were assayed using flow cytometry.

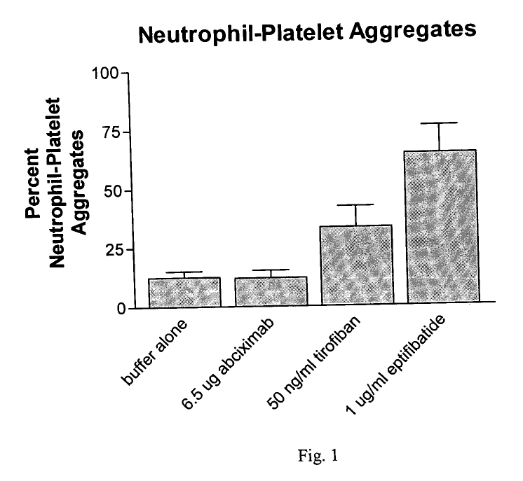

FIG. 1 shows the results of this set of experiments. In these experiments, three different GPIIb-IIIa receptor antagonists were used and the percentage of neutrophil-platelet aggregates that were formed differed with the three antagonists. Y2/51 did not augment agonist-induced leukocyte-platelet aggregation in the presence of abciximab. Agonist-induced leukocyte-platelet aggregation was augmented in the presence of tirofiban, and to a greater extent in the presence of eptifibatide.

The presence of a condition such as autoantibodies in a subject's sample that can induce thrombocytopenia or thrombotic complications in the presence of a specific GPIIb-IIIa receptor antagonist is diagnosed when there is an increase in platelet activation markers (e.g., leukocyte-platelet aggregates), in the presence of a specific GPIIb-IIIa receptor antagonist and a sub-maximal dose of platelet agonist compared to the amount of platelet activation observed in the absence of the specific GPIIb-IIIa receptor antagonist.

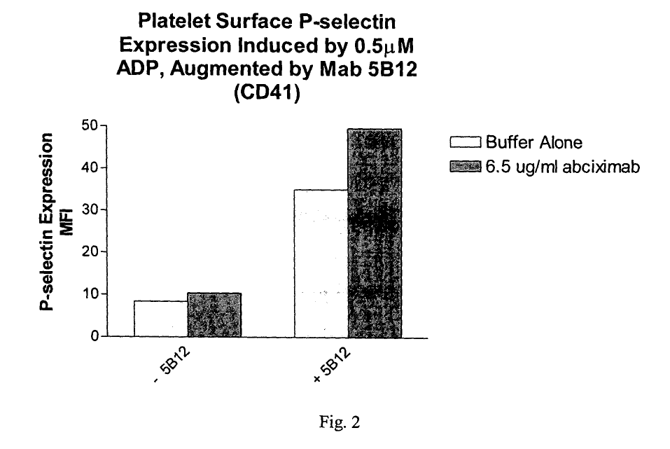

In a second set of experiments, the monoclonal antibody 5B12 (DAKO) which is GPIIb-specific was used instead of Y2/51. In these experiments, conducted essentially as described in above using Y2/51 and in Technique 3, agonist-induced platelet surface P-selectin expression was observed in the presence of abciximab (FIG. 2). Agonist-induced platelet surface P-selectin expression was not augmented in the presence of either eptifibatide or tirofiban. These data demonstrate that platelet activation in the presence of GPIIb-IIIa receptor antagonists is mediated at least in part by the platelet surface Fc receptor. These data also demonstrate the efficacy of the assay to detect augmentation of platelet activation in the presence of a GPIIb-IIIa receptor antagonist due to the presence of an antibody directed against the receptor. It is likely that this is the mechanism by which augmentation of platelet activation in the presence of GPIIb-IIIa receptor antagonists occurs in a subject, i.e., when an antibody that recognizes the receptor is present in the subject.

### Example 2

### Assay for Individuals at Risk of Becoming Thrombocytopenic or Developing Thrombotic Complications as a Result of Platelet GPIIb-IIIa Receptor Antagonist Therapy Using an FcγRII-specific Reagent

In some embodiments of the invention, assays can include the step of incubating the subject's whole blood or PRP in the presence of blocking concentrations of a FcγRII-specific reagent (e.g., CD32-specific monoclonal antibody, Mab IV.3 (Medarex)) prior to the addition of GPIIb-IIIa receptor antagonist. Experiments were performed to demonstrate this type of assay, which is generally described in Technique 4 (supra). This type of assay is used to differentiate between FcγRII-mediated immune platelet activation and other mechanisms of platelet destruction (i.e., complement-mediated).

In these experiments, platelet-rich plasma was pre-incubated with FcγRII receptor blocking monoclonal antibody (Mab IV.3, 10 μg/ml). Controls were prepared that did not contain Mab IV.3. Samples were then incubated in the presence or absence of a GPIIb-IIIa receptor antagonist (6.5 μg/ml abciximab, 50 ng/ml tirofiban, or 1 μg/ml eptifibatide), and the mixture incubated with 0.5 μM ADP and unconjugated Y2/51 or 5B12. The mean fluorescence intensity of platelet P-selectin expression in the presence of 0.5 μM ADP was measured in the presence or absence of unconjugated Y2/51 and in the presence or absence of GPIIb-IIIa receptor antagonist, and with or without an FcγRII receptor blocking monoclonal antibody (Mab IV.3).

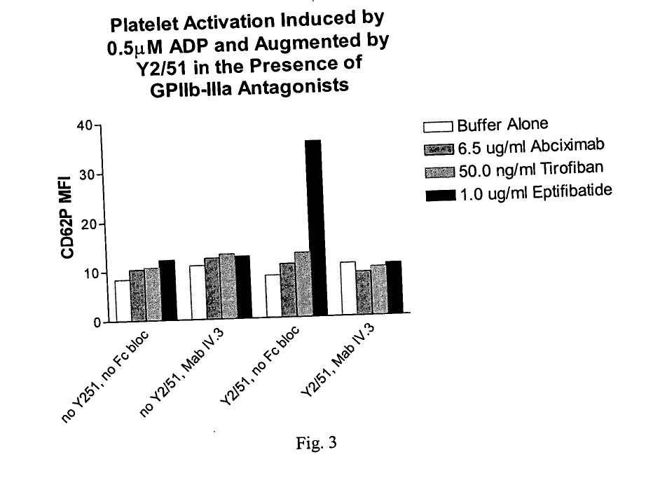

The results of these experiments demonstrated that the monoclonal antibody IV.3, directed against the FcγRII receptor on the platelet surface, inhibited the Y2/51-augmented platelet activation in the presence of eptifibatide (FIG. 3). It was also observed that augmentation by 5B12 in the presence of abciximab was inhibited (not shown). The data also confirm that that in the presence of eptifibatide and in the absence of Mab IV.3, Y2/51 augments ADP-mediated platelet activation.

Activation in the presence of FcγRII-specific blocking reagent indicates that the observed platelet activation is not mediated by FcγRII, and is therefore not likely to be mediated by an autoantibody.

Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method of determining whether an individual has thrombocytopenia or is at risk for developing thrombocytopenia as a result of GPIIb-IIIa receptor antagonist treatment, the method comprising
a. obtaining a sample comprising at least serum or plasma from the individual and platelets;
b. adding a GPIIb-IIIa receptor antagonist to the sample to form an antagonist mixture;
c. adding a sub-maximal concentration of platelet agonist to the antagonist mixture to form an assay solution; and
d. assaying platelet activation in the assay solution;
wherein, an increase in platelet activation in the assay solution compared to a reference indicates that the individual has thrombocytopenia or is at risk for developing thrombocytopenia as a result of GPIIb-IIIa receptor antagonist treatment.

2. The method of claim 1, wherein the sample comprises whole blood from the individual.

3. The method of claim 1, wherein the sample comprises serum or plasma from the individual and platelets from an ABO-compatible donor

4. The method of claim 1, further comprising adding a CD32 blocking antibody prior to step a, wherein after carrying out steps a-d, a reduction in the increase in platelet activation compared to performing the method without the CD32 blocking antibody indicates the presence of pathologic anti-platelet antibodies in the sample.

5. The method of claim 1, wherein the sample is from a human and increased platelet activation indicates that the human is at risk for developing thrombocytopenia or thrombotic complications.

6. The method of claim 1, wherein the platelet agonist is adenosine diphosphate (ADP), thrombin receptor activating peptide (TRAP), iso-TRAP, or collagen.

7. The method of claim 1, wherein the GPIIb-IIIa receptor antagonist is abciximab, eptifibatide, or tirofiban.

8. The method of claim 1, wherein platelet activation is assayed by detecting a level of platelet surface P-selectin, phosphatidylserine, or leukocyte-platelet aggregates.

9. The method of claim 8, wherein flow cytometry is used to detect the level of P-selectin, phosphatidylserine, or leukocyte-platelet aggregates in the assay solution.

10. The method of claim 1, wherein flow cytometry is used to assay platelet activation in the assay solution.

11. The method of claim 1, wherein a reagent used to detect platelet activation is added to the sample prior to adding the GPIIb-IIIa antagonist to the sample (step b).

12. The method of claim 1, wherein a reagent used to detect platelet activation is added to the antagonist mixture before the platelet agonist is added to the antagonist mixture.

13. The method of claim 1, wherein a reagent used to detect platelet activation is added to the sample with the GPIIb-IIIa antagonist or to the antagonist mixture with the platelet agonist.

14. The method of claim 1, wherein the method includes the step of determining the FcγRII genotype of platelets used in the assay.

15. The method of claim 14, wherein the platelets are from the subject.

16. The method of claim 14, wherein the platelets are from a donor.
